(19) United States
(12) Patent Application Publication
(10) Pub. No.: US 2022/0022763 A1
(43) Pub. Date: Jan. 27, 2022

(54) SYSTEMS, DEVICES, AND METHODS FOR NONINVASIVELY MONITORING BLOOD PRESSURE IN A USER

(71) Applicant: DUKE UNIVERSITY, Durham, NC (US)

(72) Inventors: Muath Bishawi, Durham, NC (US); Mark Palmeri, Durham, NC (US)

(73) Assignee: DUKE UNIVERSITY, Durham, NC (US)

(21) Appl. No.: 17/296,886

(22) PCT Filed: Dec. 3, 2019
(86) PCT No.: PCT/US2019/064135

§ 371 Date: May 25, 2021

Related U.S. Application Data
(60) Provisional application No. 62/833,133, filed on Apr. 12, 2019.
(60) Provisional application No. 62/776,727, filed on Dec. 7, 2018.
(60) Provisional application No. 62/774,419, filed on Dec. 3, 2018.

(51) Int. Cl. A61B 5/022; A61B 5/021; A61B 5/026; A61B 5/00

(52) U.S. Cl. CPC A61B 5/02233; A61B 5/02141; A61B 5/026; A61B 2560/0462; A61B 5/6829; A61B 5/6824; A61B 5/6831; A61B 5/0004

(57) ABSTRACT

A system and method for noninvasively measuring blood pressure is disclosed. In one embodiment, the system includes a monitoring cuff comprising at least one sensor implanted within the monitoring cuff, the at least one sensor being configured to detect blood flow data in a user, an occlusion cuff configured to inflate and deflate to restrict and permit blood flow in the user, the occlusion cuff being in electrical communication with the monitoring cuff, and a computing device configured to control the inflation, deflation, and pressure applied by the occlusion cuff, and the computing device being configured to record and analyze the blood flow data detected by the monitoring cuff.

### CROSS REFERENCE TO RELATED APPLICATION

This application claims priority to U.S. Provisional Patent Application No. 62/774,419, filed Dec. 3, 2018, to U.S. Provisional Patent Application No. 62/776,727, filed Dec. 7, 2018, and to U.S. Provisional Patent Application No. 62/833,133, filed Apr. 12, 2019, each of which are hereby incorporated by reference in their entirety.

### BACKGROUND

At home blood pressure monitoring systems are common. However, many at home blood pressure monitoring systems are inaccurate due to the limitation of the current method of measuring blood pressure (oscillatory based) by current noninvasive systems. The oscillometric method is dependent on pulse waves generated during constricted blood flow to accurately sense and estimate the blood pressure through a cuff. This method becomes problematic in patients on mechanical circulatory support such as having a left ventricular assist device (LVAD), specifically, patients that have little to no ventricular function (i.e., the inability to generate a pulse). The method is also limited in patients with overall low blood pressure, or a decreased pulsatility. In these patients, the blood pressure can be estimated in the hospital or clinic setting by using a Doppler device. In this method, “systolic” blood pressure maybe estimated given the higher sensitivity of the doppler to smaller variations in the sound generated going from no to low blood flow within the artery of choice. Doppler blood pressure measurements often require an additional person trained in signal acquisition and anatomical position of the artery, and the ability to simultaneously operate the pressure cuff and the doppler probe.

Thus, there is a need for a reliable, accurate noninvasive blood pressure monitor.

### SUMMARY

A system for noninvasively measuring blood pressure is disclosed. In one embodiment, the system includes a monitoring cuff comprising at least one sensor implanted within the monitoring cuff, the at least one sensor being configured to detect blood flow data in a user, an occlusion cuff configured to inflate and deflate to restrict and permit blood flow in the user, the occlusion cuff being in electrical communication with the monitoring cuff, and a computing device configured to control the inflation, deflation, and pressure applied by the occlusion cuff, and the computing device being configured to record and analyze the blood flow data detected by the monitoring cuff.

In one embodiment, the at least one sensor comprises at least one ultrasound transducer.

In another embodiment, the at least one ultrasound transducer comprises a plurality of ultrasound transducers, wherein each of the plurality of ultrasound transducers emits a signal having a frequency of about 20 KHz to 25 MHz, and wherein the system is configured to sample the plurality of ultrasound transducers to locate the ultrasound transducer with the highest frequency.

In one embodiment, the at least one sensor comprises at least one microphone.

In another aspect, the at least one microphone comprises a plurality of microphones, wherein each of the plurality of microphones detects an acoustic signal, and wherein the system is configured to sample the plurality of microphones to find the microphone with the highest signal-to-noise ratio.

In another aspect, the at least one microphone comprises a MEMS microphone.

In another aspect, a coupling pad secured to the monitoring cuff, the coupling pad comprising water or gel.

In yet another aspect, the occlusion cuff is attached to an upper arm of the user and the monitoring cuff is attached a wrist of the user.

In another embodiment, the occlusion cuff is attached to an upper leg of the user and the monitoring cuff is attached an ankle of the user.

In another aspect, the electrical communication comprises a cable.

In another aspect, the electrical communication comprises wireless communication.

In another embodiment, the system is configured to provide data to other devices to help them self-adjust based on the peripheral blood pressure or waveform characteristics.

In another aspect, the computing device comprises one of a mobile device or a computer.

In another aspect, communication with the user is provided using an application (“app”) on the computing device.

In yet another aspect, the user is implanted with a left ventricular assist device (LVAD).

In another embodiment, a method of noninvasively measuring blood pressure is disclosed. The method comprises providing an occlusion cuff positioned on a user, providing a monitoring cuff positioned on a user, the monitoring cuff comprising at least one sensor, and the monitoring cuff being in electrical communication with the occlusion cuff, inflating the occlusion cuff via a computing device, detecting the blood flow in the user via the at least one sensor, deflating the occlusion cuff via the computing device, recording the blood flow data via the computing device, and analyzing the recorded blood flow data via the computing device.

In another aspect, the at least one sensor comprises at least one ultrasound transducer.

In yet another aspect, the at least one ultrasound transducer comprises a plurality of ultrasound transducers, wherein each of the plurality of ultrasound transducers emits a signal having a frequency of about 20 KHz to 25 MHz, and wherein measuring the blood flow comprises communicating with each of the plurality of ultrasound transducers, and identifying the ultrasound transducer with the highest frequency.

In another aspect, the at least one sensor comprises at least one microphone.

In yet another aspect, wherein the at least one microphone comprises a plurality of microphones, wherein each of the plurality microphones detects an acoustic signal, and wherein measuring the blood flow comprises communicating with each of the plurality of microphones, and identifying the microphone with the highest signal-to-noise ratio.

In another aspect, a device for noninvasively measuring blood pressure is provided. The device includes a band having at least one sensor implanted therein, the at least one sensor being configured to detect blood flow data in a user, the band being configured to be in electrical communication with a computing device.

In addition to the exemplary aspects and embodiments described above, further aspects and embodiments will become apparent by reference to the drawings and by study of the following detailed description.

### BRIEF DESCRIPTION OF THE DRAWINGS

FIGS. 1A and 1B are diagrams showing a noninvasive blood pressure monitoring device in accordance with one embodiment of the present disclosure.

FIGS. 2A and 2B are diagrams showing a noninvasive blood pressure monitoring device incorporating a plurality of sensors in accordance with one embodiment of the present disclosure.

FIG. 3 is a diagram showing a noninvasive blood pressure monitoring device placed on a wrist of a subject.

FIG. 4 is a flow chart illustrating an example method of the present disclosure.

FIG. 5 is a diagram showing a noninvasive blood pressure monitoring device in accordance with another embodiment of the present disclosure.

FIG. 6 depicts a block diagram of a computing device and a computer network, according to an example implementation.

FIGS. 7-18 show test results obtained by testing of a prototype according to an example embodiment of the present disclosure.

### DETAILED DESCRIPTION

For the purposes of promoting an understanding of the principles of the disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

Unless otherwise defined, all technical terms used herein have the same meaning as commonly understood by one of ordinary skill in the art to which this disclosure belongs.

The present disclosure provides, in part, a noninvasive method to measure blood pressure that utilizes acoustic sensor technology to evaluate blood flow before and after occlusion of the artery.

A blood pressure monitor having an occlusion cuff and a monitoring cuff, both cuffs being in communication with a computing device, is disclosed. The process of signal acquisition, recording, and interpretation is automated through the use of one or more sensors implanted in the monitoring cuff. The sensors may be acoustic sensors such as an ultrasound transducer or a microphone. Once the signal is found, the system automatically inflates and deflates the occlusion cuff giving an estimation of the blood pressure. The blood pressure is automatically recorded by the computing device, such as into a mobile app, and time stamped. Additional data to be stored includes the signal quality obtained by the sensor to give a degree of confidence of the blood pressure reading. Readings over time can be analyzed and trends recorded. Additional alerts can be built and customized.

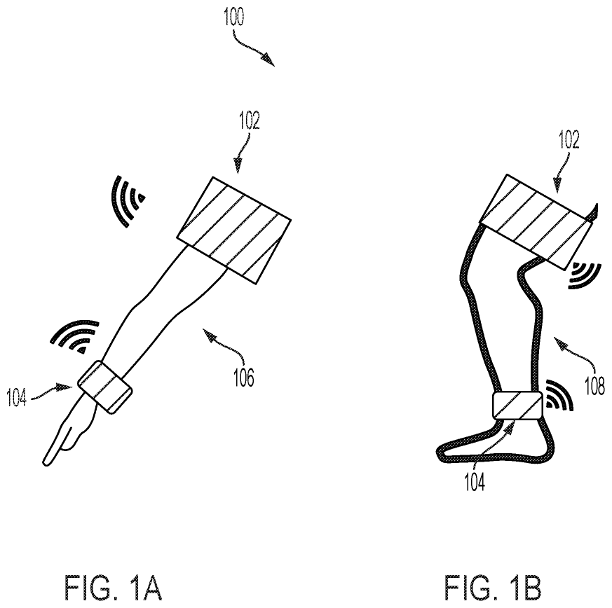

FIGS. 1A and 1B show a system 100 for noninvasively measuring blood pressure in a user, patient, or subject. The system 100 includes an occlusion cuff 102 and a monitoring cuff 104. In some embodiments, the occlusion cuff 102 is attached to the upper arm 106 of the subject and the monitoring cuff 104 is attached the wrist of the subject. In another embodiment, the occlusion cuff 102 is attached to the upper leg 108 of a subject and the monitoring cuff 104 is attached to the ankle of the subject. The cuff size may vary depending upon placement.

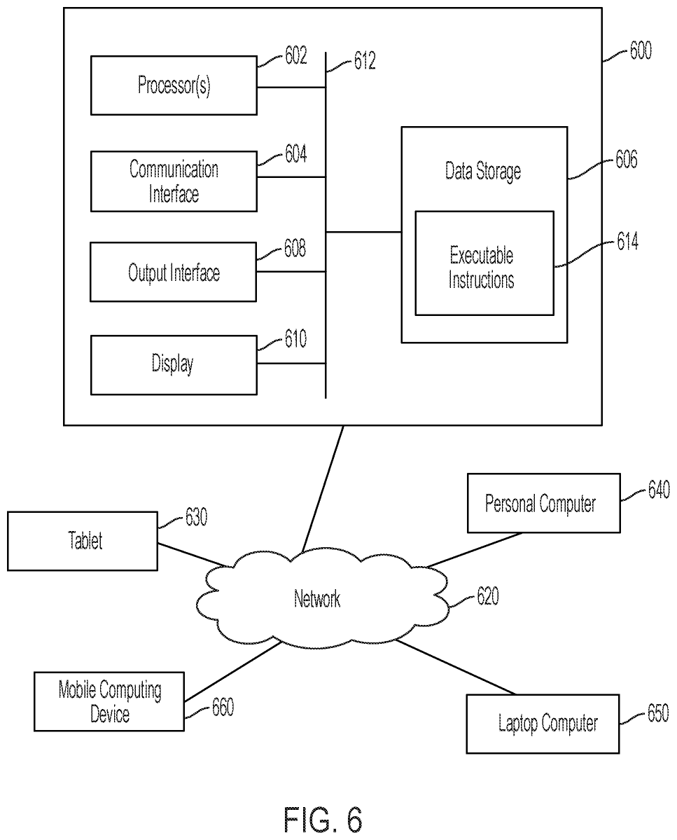

The occlusion cuff 102 includes an inflatable bladder, such as in a typical blood pressure cuff. The occlusion cuff 102 can be placed around a subject's upper arm or upper leg near an artery. Pressure can be increased and decreased in the bladder in order to stop and start blood flow in an artery. The occlusion cuff 102 further comprises an electrical component for electrically communicating with the monitoring cuff 104 and a computing device. In some embodiments, the inflation and deflation of the occlusion cuff 102 can be controlled by the computing device. The computing device is described in detail below with reference to FIG. 6.

The monitoring cuff 104 may be configured as a generally circular cuff that can be placed around a subject's wrist or ankle near an artery to measure blood flow. In some embodiments, the monitoring cuff 104 may include a latching mechanism (not shown) to enable the cuff to open and close around the subject. In some embodiments, the monitoring cuff 104 is a single unitary band without a latching mechanism.

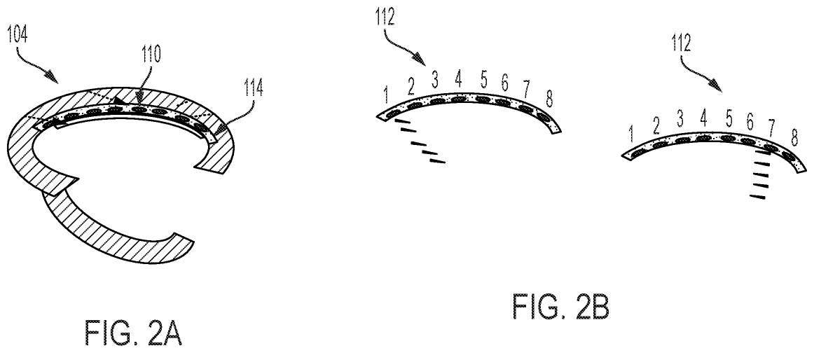

The monitoring cuff 104 further includes one or more sensors 110 to measure blood flow in an artery of a subject, which is described in more detail below with respect to FIGS. 2A and 2B. In some embodiments, the at least one sensor comprises a plurality or array of sensors located within the monitoring cuff 104. The monitoring cuff 104 further comprises an electrical component for electrically communicating with the occlusion cuff 102 and the computing device.

It should be understood that in some embodiments, the device includes only a monitoring cuff or band 104 and no occlusion cuff is present. The monitoring cuff 104 may be placed on the ankle, wrist, or any other artery (carotid artery, for example) of a patient, or intraoperatively to detect the blood flow. The monitoring cuff includes one or more sensors 110 as described above.

In some embodiments, the electrical communication between the occlusion cuff 102 and the monitoring cuff 104 comprises a cable. In another embodiment, the electrical communication comprises wireless communication. In some embodiments, the wireless communication is selected from the group consisting of WiFi, Bluetooth, satellite communication, infrared communication, radio communication, microwave communication, cellular communication, and combinations thereof.

Referring to FIGS. 2A and 2B, the monitoring cuff 104 comprises one or more sensors 110 for sensing blood flow. In some embodiments, a plurality or array 112 of sensors 110 are positioned within the monitoring cuff 104. Although eight (8) sensors are shown in the Figures, it should be understood that more or less sensors may be used. In one example, the sensor(s) 110 may comprise ultrasound transducers. The ultrasound transducers emit a signal having a frequency of about 20 KHz to about 25 MHz. In some embodiments, the computing device is configured to sample the array of ultrasound transducers to locate the ultrasound transducer with the highest frequency, which corresponds to the best signal-to-noise ratio in the artery.

In another example, the sensor(s) 110 may comprise microphones. In some embodiments, the microphones may be micro-electromechanical systems (MEMS) microphones. In other embodiments, the microphones may be piezoelectric elements, capacitive diaphragms (e.g., condenser microphone), and inductive coils (e.g., dynamic microphones). The microphones detect acoustic signals from blood flow in the artery and convert them into an electrical signal. This is done through the transduction of the pressure waves associated with the acoustic sound into a mechanical translation of deformation of the microphone's transductive element, resulting in the generation of a voltage or current signal that can then be processed downstream in hardware or software to detect the blood flow. In some embodiments, the computing device is configured to sample the plurality or array of microphones to locate the microphone with the highest signal-to-noise ratio.

The signal obtained by the monitoring cuff 104 is therefore much more accurate than the measurements obtained in typical blood pressure monitors since the blood flow in the artery is being directly measured rather than being dependent upon vibrations.

In some embodiments, the monitoring cuff 104 further includes a coupling pad 114 secured thereto. The coupling pad 114 may improve the interface between the sensor(s) 110 and the skin of a subject. In one example, the coupling pad 114 may comprise a water or gel.

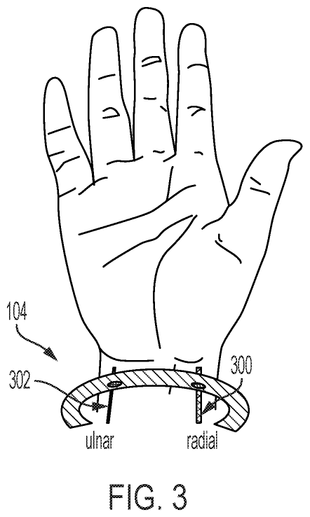

Referring to FIG. 3, the monitoring cuff 104 is placed so the sensor(s) 110 are in line with the radial artery 300 and ulnar artery 302. In the case where the monitoring cuff 104 is positioned on the upper leg, the monitoring cuff 104 is placed so the sensor(s) 110 are in line with the popliteal artery and posterior tibial artery. In some embodiments, the monitoring cuff 104 may include a drawing or other indication regarding the correct placement on the body. In some embodiments, a user can confirm correct placement of the monitoring cuff 104 via an instruction manual, or through an indication on the computing device.

In some embodiments, the monitoring cuff 104 is made of a material that is acoustically attenuating to reduce ambient noise, for example. It should be understood that any suitable material may be used for the monitoring cuff 104.

In some embodiments, the systems and devices according to the present disclosure also provide data to other devices to help them self-adjust based on the peripheral blood pressure or waveform characteristics. For instance, the blood pressure system can provide data back to an automated circulatory support device (e.g., LVAD, balloon pump, etc.), or a pace maker to help adjust flow or heart rate based on the current peripheral blood pressure.

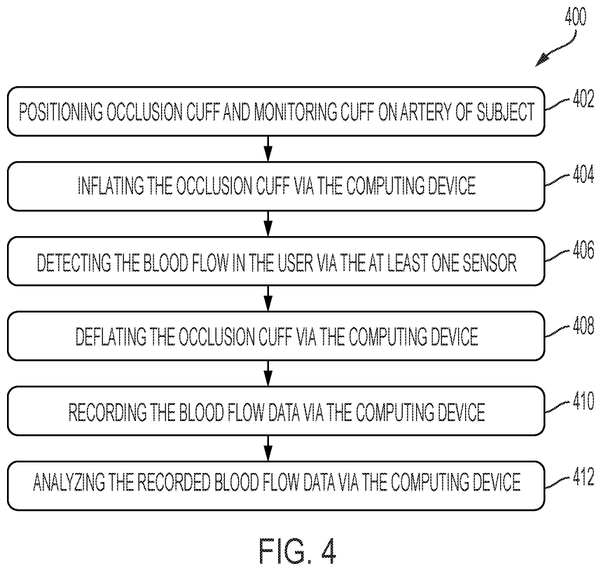

Referring to FIG. 4, a flow chart illustrating an example method 400 of the present disclosure. Each block or portions of each block in FIG. 4, and within other processes and methods disclosed herein, may be performed by or in accordance with the system described above with respect to FIGS. 1-3. Alternative implementations are included within the scope of the examples of the present disclosure in which functions may be executed out of order from that shown or discussed, including substantially concurrent or in reverse order, depending on the functionality involved, as would be understood by those reasonably skilled in the art.

Method 400 begins at block 402, where the blood pressure system 100 is positioned on a user, subject, or patient. The occlusion cuff 102 is attached to the upper arm or upper leg of the subject, and the monitoring cuff 104 is attached the wrist or ankle of the subject. The occlusion cuff 102 electronically communicates with the monitoring cuff 104 either wirelessly or with a cable, for example. The occlusion cuff 102 and monitoring cuff 104 also each electronically communicate with a computing device 600.

Next, at block 404, the computing device 600 begins inflation of the occlusion cuff 102. Next, at block 406, the blood flow in the user is detected via the at least one sensor 110 in the monitoring cuff 104. Then, at block 408, the computing device deflates the occlusion cuff 102. At block 410, the computing device 600 and/or monitoring cuff 104 records when the signal is lost during inflation of the occlusion cuff 102 as the systolic pressure, and when the signal is first regained during deflation of the occlusion cuff 102 as the diastolic pressure. In cases with continuous flow, only a mean pressure is recorded. Finally, at block 412, the computing device 600 analyzes the recorded blood flow data. The analysis may compare the signal versus noise and apply a filter as appropriate to increase the sensitivity of detecting the presence of blood flow.

In some examples, prior to inflation of the occlusion cuff 102, and once placement of the monitoring cuff 104 is confirmed to be correct, the monitoring cuff 104 may provide the subject with the “OK” to begin inflation of the occlusion cuff 102 by pressing “start,” for example. In some embodiments, the communication with the subject is provided using an application (“app”) on the computing device (e.g., a mobile device such as an iphone/android phone, or tablet, a laptop, a computer, etc.). In some examples, if the placement of the monitoring cuff 104 is determined to be incorrect, an alert can be provided to the user to reposition the cuff 104.

The app can also be used by the subject to view trends and provide alerts. In some embodiments, the trends and alerts may include changes of blood pressure over time, indicating changes in the patient's hemodynamics. For example, a heart failure patient might have increases of blood pressure over time (on average), indicating they might be gaining too much water weight requiring increased dosage of medications.

In some embodiments, the app and/or computing device may connect to a hospital network, or directly upload the blood pressure data to a central data portal or the “cloud.”

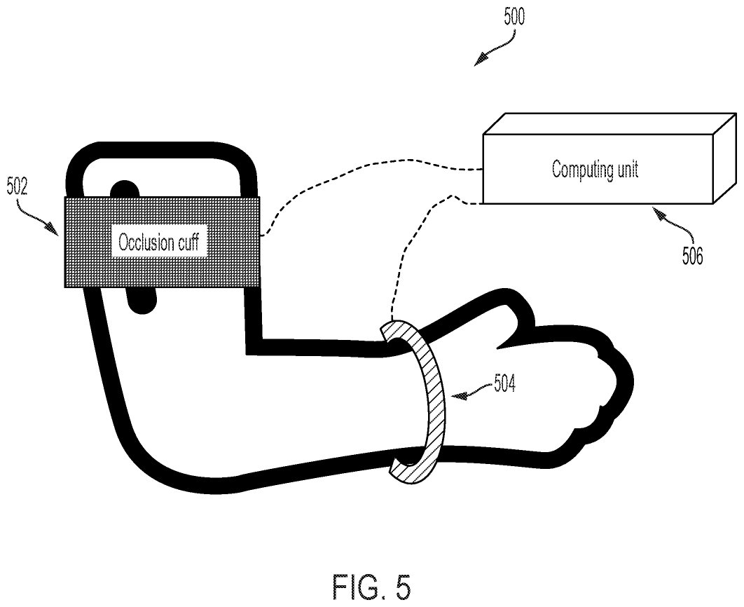

FIG. 5 shows an alternate embodiment of a noninvasive blood pressure system 500. As shown in FIG. 5, a blood pressure cuff 502 is secured to the upper arm of a patient to occlude blood flow in the brachial artery. A sleeve 504 with several sensors, such as MEMS microphones, is then placed on the forearm to locate the sound of turbulent blood flow as the artery is occluded. Hardware for other electrical components may be contained in a PCB board, and a computing unit or microprocesser 506 may be used for managing software of the device. These components may be contained externally. When the sound of turbulent blood flow is detected, a clinician is alerted to record the blood pressure reading as a more accurate MAP for the patient.

FIG. 6 is a block diagram illustrating an example of the computing device 600, according to an example implementation, that is configured to interface with occlusion cuff 102 and monitoring cuff 104, either directly or indirectly. The computing device 600 may be used to perform functions of methods shown in FIG. 4 and described throughout the disclosure. In particular, computing device 600 can be configured to perform one or more functions, including detecting, recording, and analyzing blood flow, for example. The computing device 600 has a processor(s) 602, and also a communication interface 604, data storage 606, an output interface 608, and a display 610 each connected to a communication bus 612. The computing device 600 may also include hardware to enable communication within the computing device 600 and between the computing device 600 and other remote devices. The hardware may include transmitters, receivers, and antennas, for example.

The communication interface 604 may be a wireless interface and/or one or more wired interfaces that allow for both short-range communication and long-range communication to one or more networks 620 or to one or more remote computing devices (e.g., a tablet 630, a personal computer 640, a laptop computer 650 and a mobile computing device 660, for example). Such wireless interfaces may provide for communication under one or more wireless communication protocols, such as Bluetooth, Wi-Fi (e.g., an institute of electrical and electronic engineers (IEEE) 802.11 protocol), Long-Term Evolution (LTE), cellular communications, near-field communication (NFC), and/or other wireless communication protocols. Such wired interfaces may include Ethernet interface, a Universal Serial Bus (USB) interface, or similar interface to communicate via a wire, a twisted pair of wires, a coaxial cable, an optical link, a fiber-optic link, or other physical connection to a wired network. Thus, the communication interface 604 may be configured to receive input data from one or more devices, and may also be configured to send output data to other devices.

The communication interface 604 may also include a user-input device, such as a keyboard, a keypad, a touch screen, a touch pad, a computer mouse, a track ball and/or other similar devices, for example.

The data storage 606 may include or take the form of one or more computer-readable storage media that can be read or accessed by the processor(s) 602. The computer-readable storage media can include volatile and/or non-volatile storage components, such as optical, magnetic, organic or other memory or disc storage, which can be integrated in whole or in part with the processor(s) 602. The data storage 606 is considered non-transitory computer readable media. In some examples, the data storage 606 can be implemented using a single physical device (e.g., one optical, magnetic, organic or other memory or disc storage unit), while in other examples, the data storage 606 can be implemented using two or more physical devices.

The data storage 606 thus is a non-transitory computer readable storage medium, and executable instructions 614 are stored thereon. The instructions 614 include computer executable code. When the instructions 614 are executed by the processor(s) 602, the processor(s) 602 are caused to perform functions. Such functions include, but are not limited to, detecting, recording, and analyzing blood flow.

The processor(s) 602 may be a general-purpose processor or a special purpose processor (e.g., digital signal processors, application specific integrated circuits, etc.). The processor(s) 602 may receive inputs from the communication interface 604, and process the inputs to generate outputs that are stored in the data storage 606 and output to the display 610. The processor(s) 602 can be configured to execute the executable instructions 614 (e.g., computer-readable program instructions) that are stored in the data storage 606 and are executable to provide the functionality of the computing device 600 described herein.

The output interface 608 outputs information to the display 610 or to other components as well. Thus, the output interface 608 may be similar to the communication interface 604 and can be a wireless interface (e.g., transmitter) or a wired interface as well. The output interface 608 may send commands to one or more controllable devices, for example.

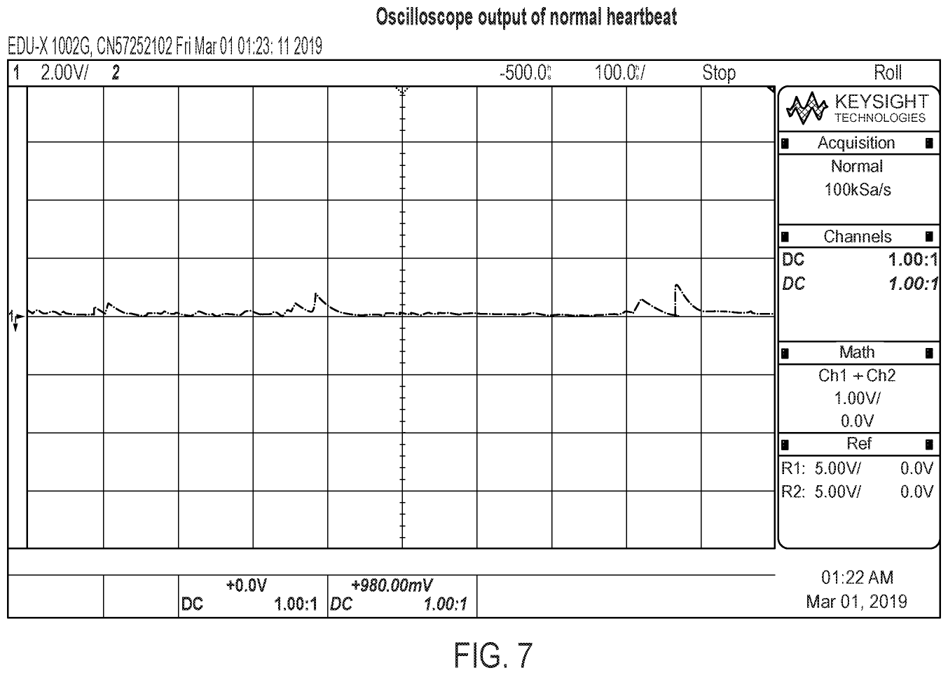

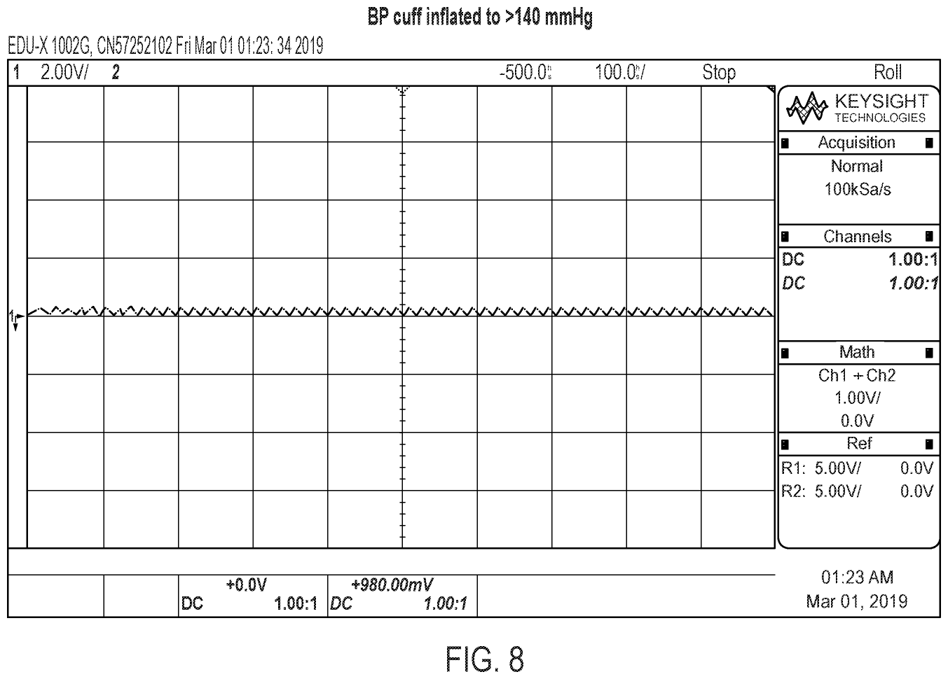

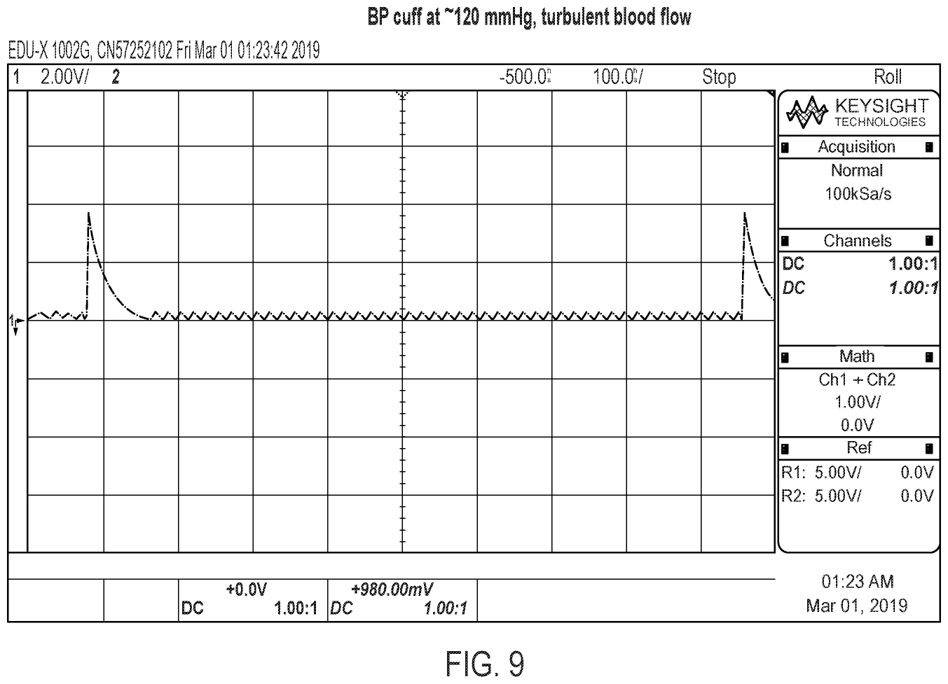

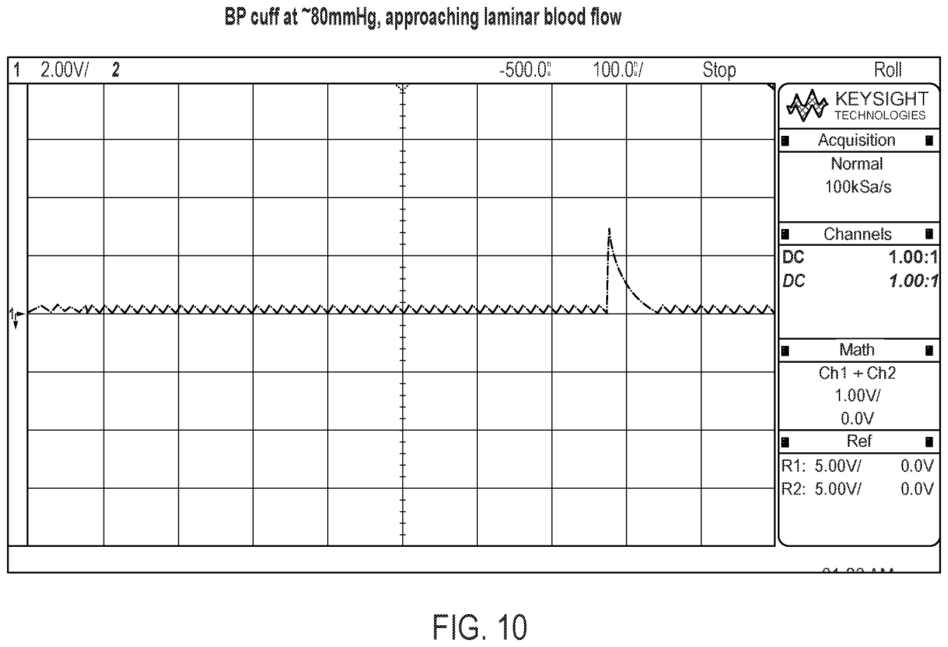

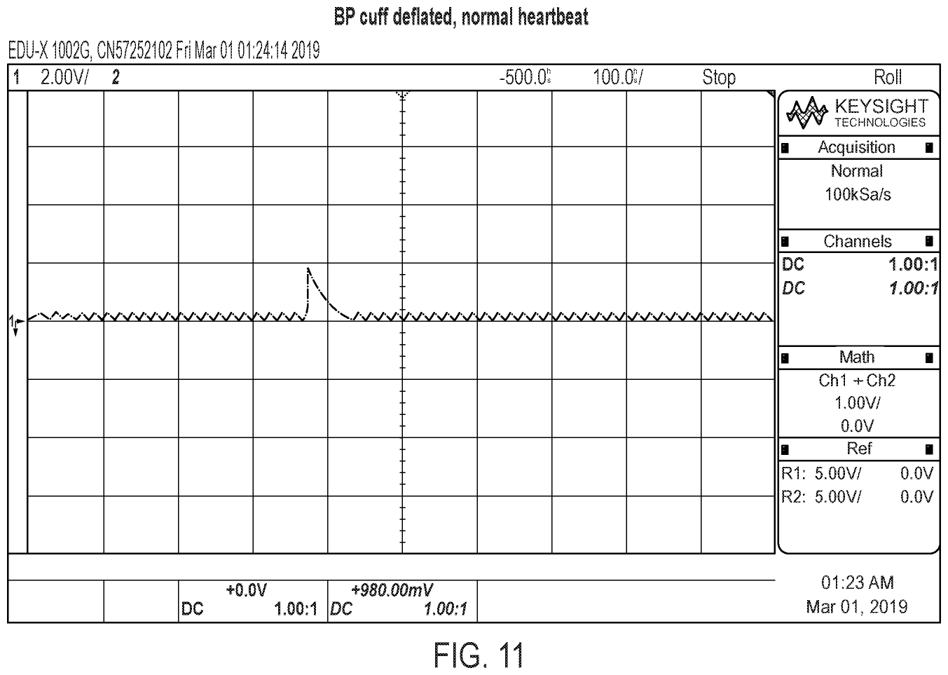

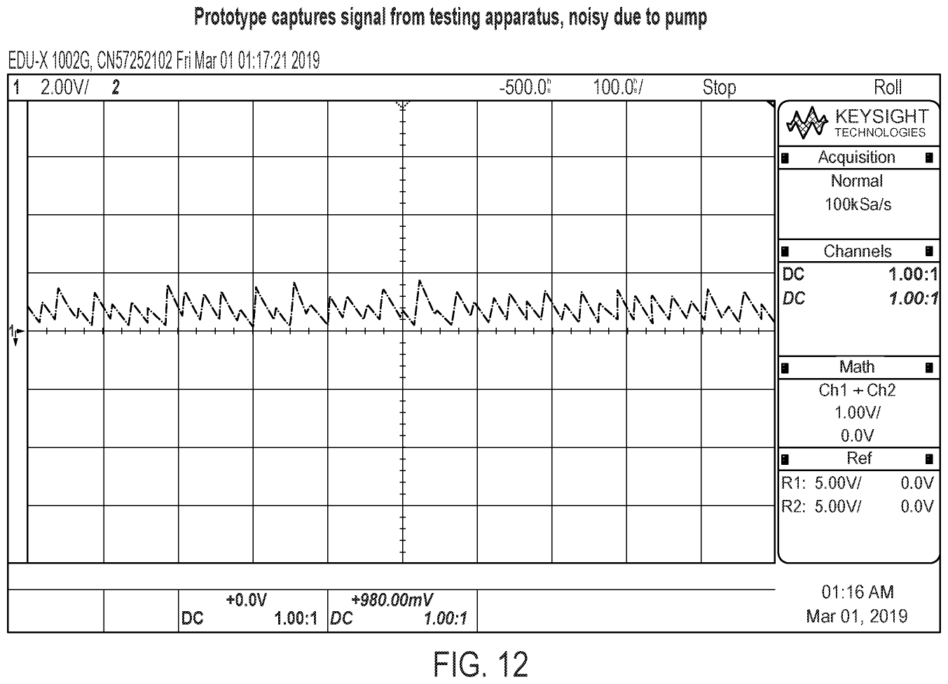

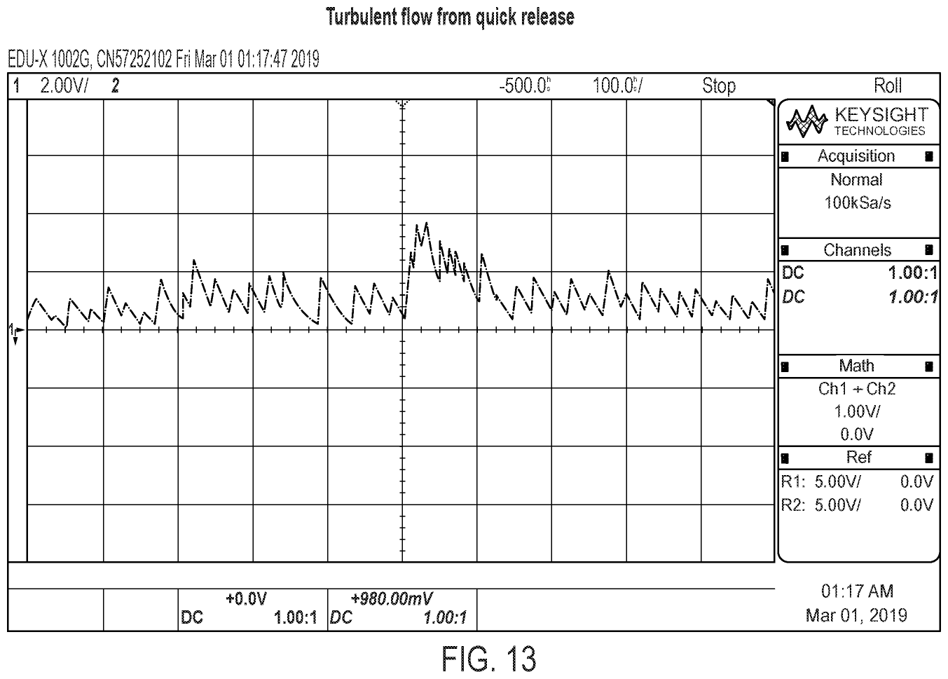

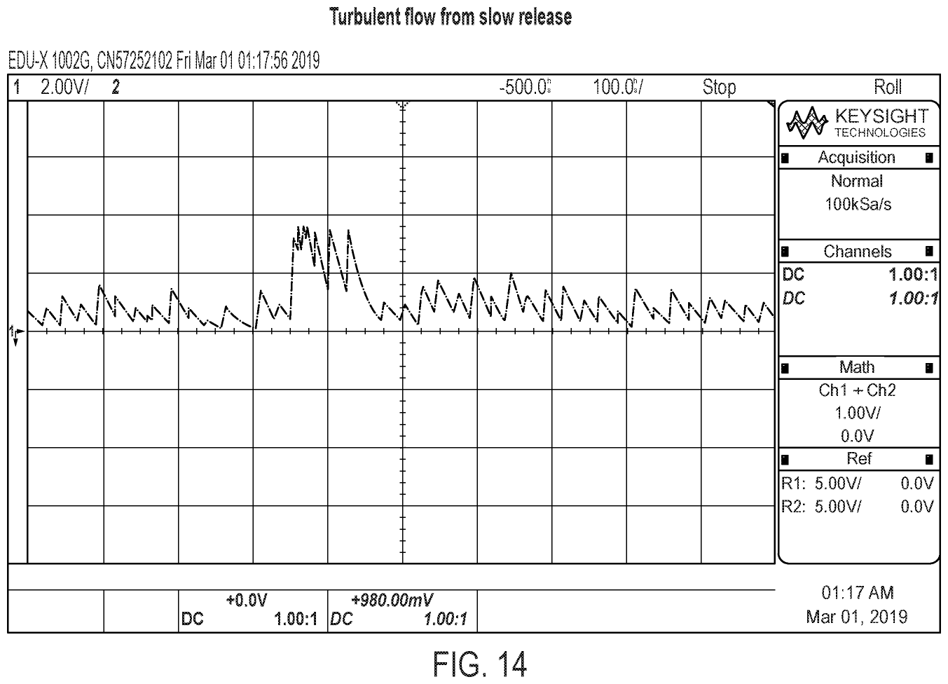

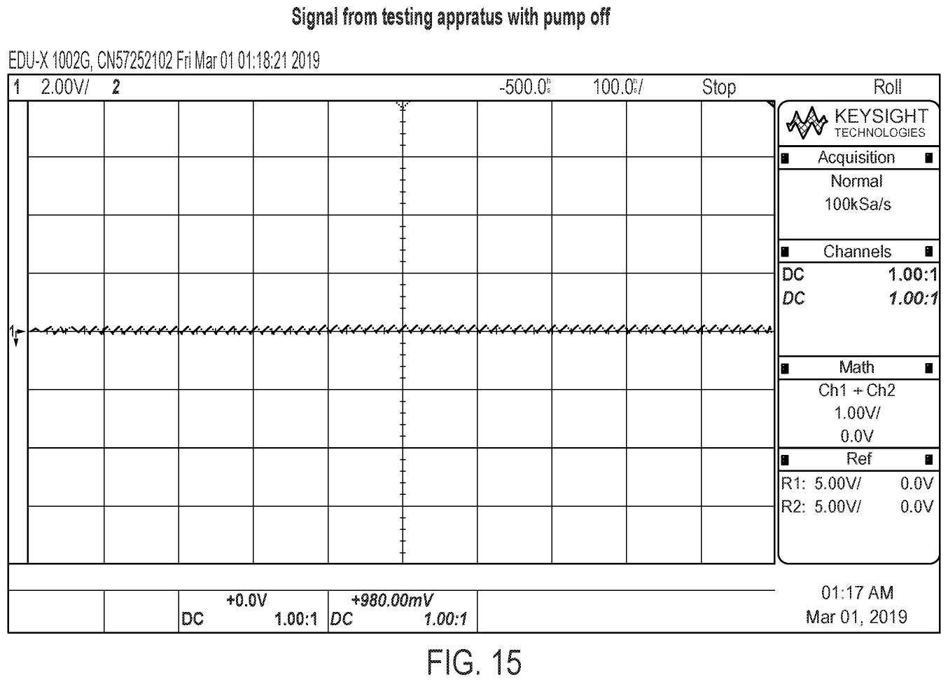

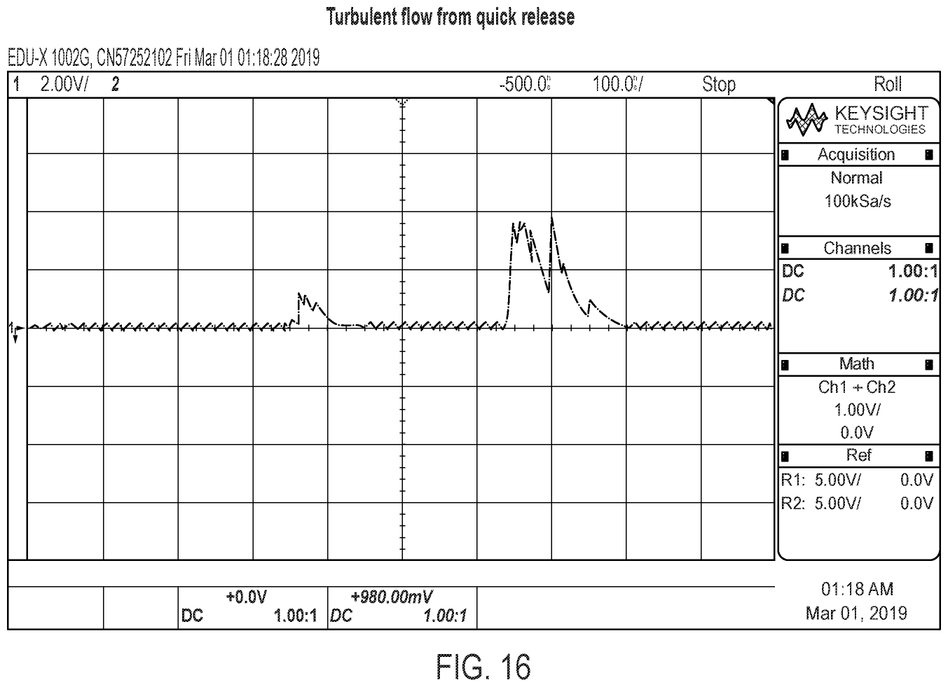

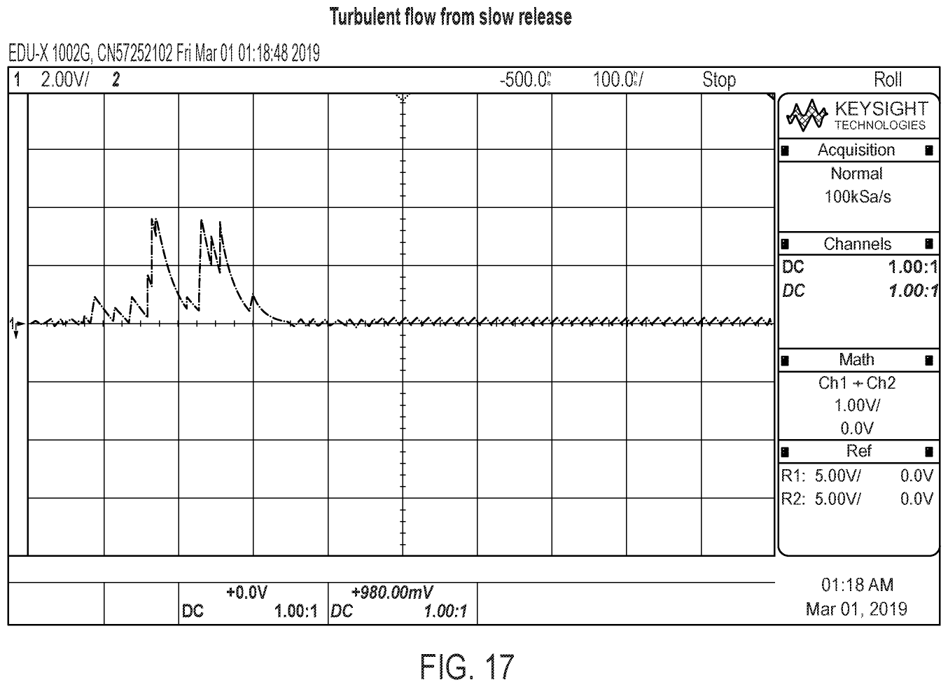

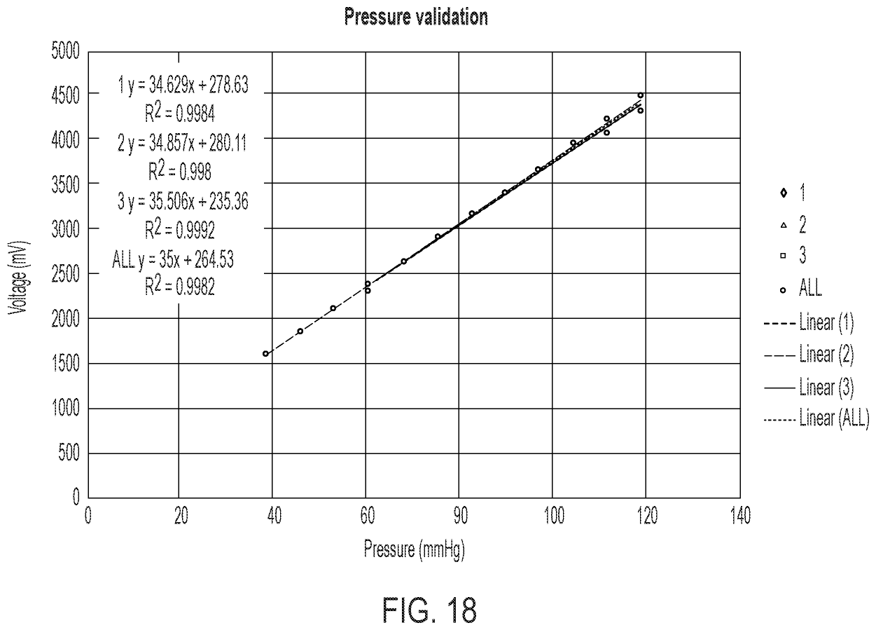

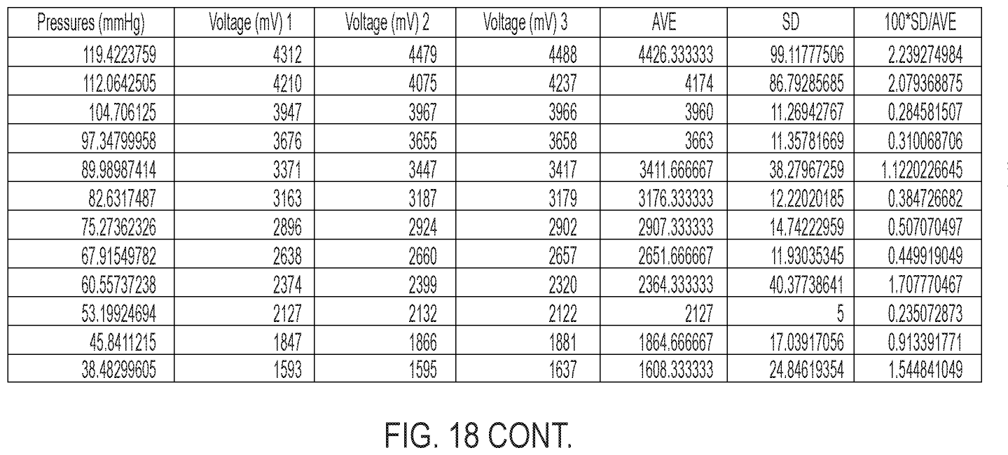

FIGS. 7-18 show test results obtained by testing of a prototype device in accordance with an example embodiment of the disclosure.

One skilled in the art will readily appreciate that the present application is well adapted to carry out the objects and obtain the ends and advantages mentioned, as well as those inherent therein. The present disclosure described herein is presently representative of preferred embodiments, are exemplary, and are not intended as limitations on the scope of the disclosure. Changes therein and other uses will occur to those skilled in the art which are encompassed within the spirit as defined by the scope of the claims.

While a number of exemplary aspects and embodiments have been discussed above, those of skill in the art will recognize that still further modifications, permutations, additions and sub-combinations thereof of the features of the disclosed embodiments are still possible. It is therefore intended that the following appended claims and claims hereafter introduced are interpreted to include all such modifications, permutations, additions and sub-combinations as are within their true spirit and scope.

1. A system for noninvasively measuring blood pressure, the system comprising:
a monitoring cuff comprising at least one sensor implanted within the monitoring cuff, the at least one sensor being configured to detect blood flow data in a user;
an occlusion cuff configured to inflate and deflate to restrict and permit blood flow in the user, the occlusion cuff being in electrical communication with the monitoring cuff; and
a computing device configured to control the inflation, deflation, and pressure applied by the occlusion cuff, and the computing device being configured to record and analyze the blood flow data detected by the monitoring cuff.

2. The system of claim 1, wherein the at least one sensor comprises at least one ultrasound transducer.

3. The system of claim 2, wherein the at least one ultrasound transducer comprises a plurality of ultrasound transducers, wherein each of the plurality of ultrasound transducers emits a signal having a frequency of about 20 KHz to 25 MHz, and wherein the system is configured to sample the plurality of ultrasound transducers to locate the ultrasound transducer with the highest frequency.

4. The system of claim 1, wherein the at least one sensor comprises at least one microphone.

5. The system of claim 4, wherein the at least one microphone comprises a plurality of microphones, wherein each of the plurality of microphones detects an acoustic signal, and wherein the system is configured to sample the plurality of microphones to find the microphone with the highest signal-to-noise ratio.

6. (canceled)

7. The system of claim 1, further comprising a coupling pad secured to the monitoring cuff, the coupling pad comprising water or gel.

8. The system of claim 1, wherein the occlusion cuff is operable to be attached to an upper arm of the user and the monitoring cuff is operable to be attached a wrist of the user or the occlusion cuff is operable to be attached to an upper leg of the user and the monitoring cuff is operable to be attached an ankle of the user.

9. (canceled)

10. The system of claim 1, wherein the electrical communication comprises a cable.

11. The system of claim claim 1, wherein the electrical communication comprises wireless communication.

12. The system of claim 1, wherein the system is configured to provide data to other devices to help them self-adjust based on the peripheral blood pressure or waveform characteristics.

13. The system of claim 1, wherein the computing device comprises one of a mobile device or a computer.

14. The system of claim 1, wherein communication with the user is provided using an application (“app”) on the computing device.

15. The system of claim 1, wherein the user is implanted with a left ventricular assist device (LVAD).

16. A method for noninvasively measuring blood pressure, the method comprising:
providing an occlusion cuff positioned on a user;
providing a monitoring cuff positioned on a user, the monitoring cuff comprising at least one sensor, and the monitoring cuff being in electrical communication with the occlusion cuff;
inflating the occlusion cuff via a computing device;
detecting the blood flow in the user via the at least one sensor;
deflating the occlusion cuff via the computing device;
recording the blood flow data via the computing device; and
analyzing the recorded blood flow data via the computing device.

17. The method of claim 16, wherein the at least one sensor comprises at least one ultrasound transducer.

18. The method of claim 17, wherein the at least one ultrasound transducer comprises a plurality of ultrasound transducers, wherein each of the plurality of ultrasound transducers emits a signal having a frequency of about 20 KHz to 25 MHz, and wherein measuring the blood flow comprises communicating with each of the plurality of ultrasound transducers, and identifying the ultrasound transducer with the highest frequency.

19. The method of claim 16, wherein the at least one sensor comprises at least one microphone.

20. The method of claim 19, wherein the at least one microphone comprises a plurality of microphones, wherein each of the plurality microphones detects an acoustic signal, and wherein measuring the blood flow comprises communicating with each of the plurality of microphones, and identifying the microphone with the highest signal-to-noise ratio.

21. The method of claim 16, wherein the occlusion cuff is attached to an upper arm of the user and the monitoring cuff is attached a wrist of the user or the occlusion cuff is attached to an upper leg of the user and the monitoring cuff is attached an ankle of the user.

22. (canceled)

23. A device for noninvasively measuring blood pressure comprising:
a band having at least one sensor implanted therein, the at least one sensor being configured to detect blood flow data in a user, the band being configured to be in electrical communication with a computing device.
